(19) United States
(12) Plant Patent Application Publication
(10) Pub. No.: US 2016/0324056 P1
(43) Pub. Date: Nov. 3, 2016

(54) LITJAC

(50) ;

(71) Applicant: Adrian Bloom, Diss (GB)

(72) Inventor: Adrian Bloom, Diss (GB)

(21) Appl. No.: 14/545,371

(22) Filed: Apr. 28, 2015

(51) Int. Cl. A01H 5/00

(52) U.S. Cl. PLT/412

(57) ABSTRACT

A new and distinct cultivar of Brunnera plant named ‘Litjac’, characterized by its compact, upright and uniformly mounded plant habit; freely clumping habit; relatively small leaves that are light green in color with greyed green-colored intervenal areas; and good container and garden performance.

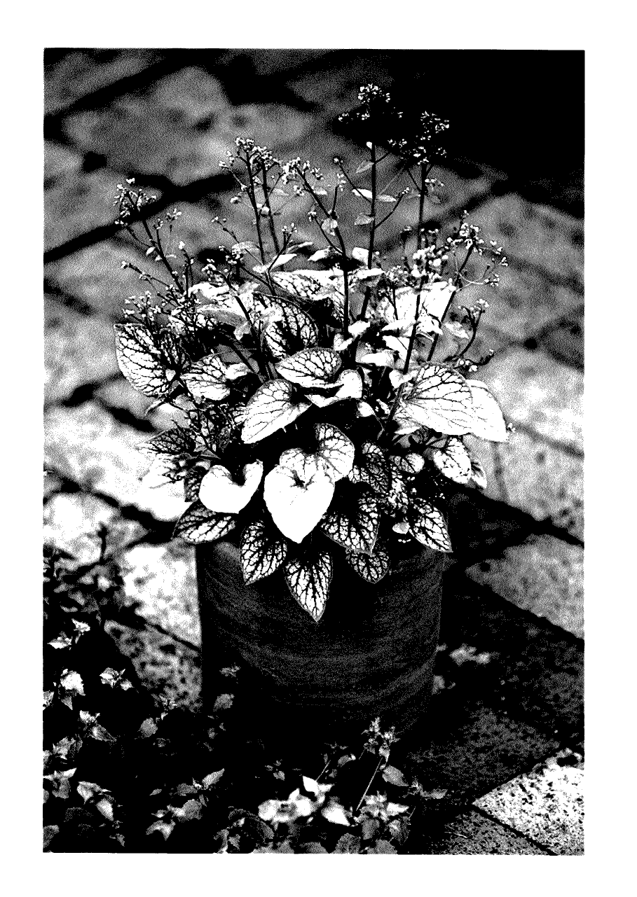

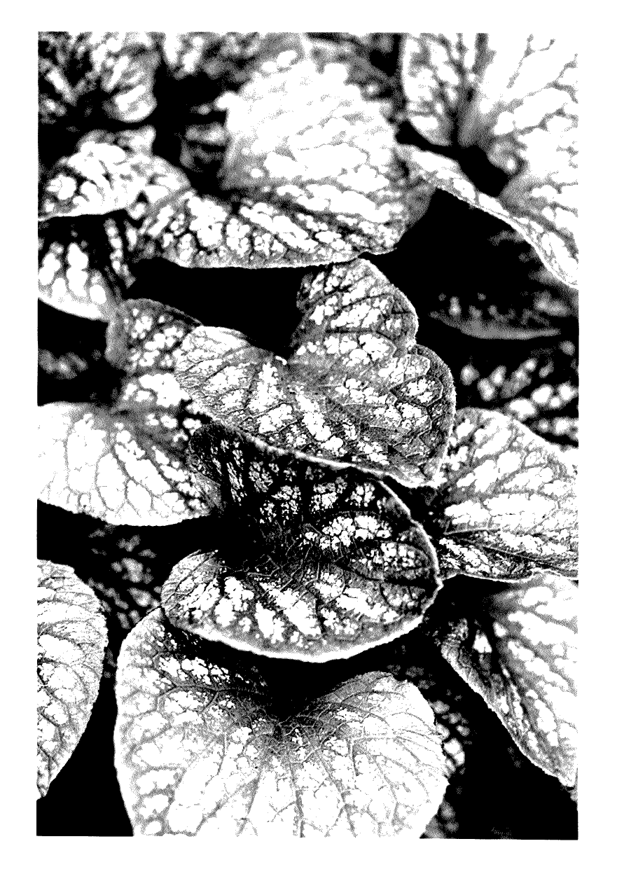

### BOTANICAL DESIGNATION

Brunnera macrophylla

### CULTIVAR DENOMINATION

‘LITJAC’

### BACKGROUND OF THE INVENTION

The present invention relates to a new and distinct cultivar of Brunnera, botanically known as Brunnera macrophylla and hereinafter referred to by the name ‘Litjac’.

The new Brunnera plant is a product of a planned breeding program conducted by the Inventor in Bressingham, Diss, Norfolk, United Kingdom. The objective of the breeding program is to create new Brunnera plants with small leaves.

The new Brunnera plant originated from an open-pollination in Bressingham, Diss, Norfolk, United Kingdom in May, 2005 of an unnamed selection of Brunnera macrophylla, not patented, as the female, or seed, parent with an unknown selection of Brunnera macrophylla as the male, or pollen, parent. The new Brunnera plant was discovered and selected by the Inventor as a single plant from within the progeny of the stated open-pollination in a controlled outdoor nursery environment in Bressingham, Diss, Norfolk, United Kingdom in May, 2005.

Asexual reproduction of the new Brunnera plant by divisions in a controlled greenhouse environment in Bressingham, Diss, Norfolk, United Kingdom since the spring of 2006 has shown that the unique features of this new Brunnera plant are stable and reproduced true to type in successive generations.

### SUMMARY OF THE INVENTION

The new Brunnera plant has not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Litjac’. These characteristics in combination distinguish ‘Litjac’ as a new and distinct Brunnera plant:

1. Compact, upright and uniformly mounded plant habit.

2. Freely clumping habit.

3. Relatively small leaves that are light green in color with greyed green-colored intervenal areas.

4. Good container and garden performance.

Plants of the new Brunnera differ from plants of the female parent selection primarily in leaf size as plants of the new Brunnera have smaller leaves than plants of the female parent selection.

Plants of the new Brunnera can be compared to plants of the Brunnera macrophylla ‘Jack Frost’, disclosed in U.S. Plant Pat. No. 13,859. In side-by-side comparisons conducted in Bressingham, Diss, Norfolk, United Kingdom, plants of the new Brunnera differed from plants of ‘Jack Frost’ in the following characteristics:

1. Plants of the new Brunnera were more compact than plants of ‘Jack Frost’.

2. Plants of the new Brunnera were less vigorous than plants of ‘Jack Frost’.

3. Plants of the new Brunnera had smaller leaves than plants of ‘Jack Frost’.

4. Plants of the new Brunnera and ‘Jack Frost’ differed in leaf color as plants of ‘Jack Frost’ had more pronounced greyed green-colored areas than plants of the new Brunnera.

5. Plants of the new Brunnera had smaller flowers than plants of ‘Jack Frost’.

Plants of the new Brunnera can also be compared to plants of the Brunnera macrophylla ‘Silver Wings’, disclosed in U.S. Plant Pat. No. 13,706. In side-by-side comparisons conducted in Bressingham, Diss, Norfolk, United Kingdom, plants of the new Brunnera differed from plants of ‘Silver Wings’ in the following characteristics:

1. Plants of the new Brunnera were more compact than plants of ‘Silver Wings’.

2. Plants of the new Brunnera were less vigorous than plants of ‘Silver Wings’.

3. Plants of the new Brunnera had smaller leaves than plants of ‘Silver Wings’.

4. Plants of the new Brunnera and ‘Silver Wings’ differed in leaf color as plants of ‘Silver Wings’ had greyed green-colored leaves with thin cream-colored margins and a silver overlay.

5. Plants of the new Brunnera had smaller flowers than plants of ‘Silver Wings’.

### BRIEF DESCRIPTION OF THE PHOTOGRAPHS

The accompanying colored photographs illustrate the overall appearance of the new Brunnera showing the colors as true as it is reasonably possible to obtain in colored reproductions of this type. Colors in the photographs may differ slightly from the color values cited in the detailed botanical description which accurately describe the colors of the new Brunnera. The photograph on the first sheet comprises a side perspective view of a typical flowering plant of ‘Litjac’ grown in a container. The photograph on the second sheet is a close-up view of a typical plant of ‘Litjac’.

### DETAILED BOTANICAL DESCRIPTION

The aforementioned photographs and following observations, measurements and values describe plants grown during the spring, summer and autumn in five-liter containers in an outdoor nursery in Bressingham, Diss, Norfolk, United Kingdom and under cultural practices typical of commercial Brunnera production. During the production of the plants, day temperatures ranged from 18° C. to 22° C. and night temperatures ranged from 6° C. to 15° C. Plants used in the photographs and description were three years old. In the description, color references are made to The Royal Horticultural Society Colour Chart, 2000 Edition, except where general terms of ordinary dictionary significance are used.
Botanical classification: Brunnera macrophylla ‘Litjac’.
Parentage:
   Female, or seed, parent.—Unnamed selection of Brunnera macrophylla, not patented.
   Male, or pollen, parent.—Unknown selection of Brunnera macrophylla, not patented.
Propagation:
   Type.—By divisions.
   Time to initiate roots, summer.—About two weeks at temperatures ranging from about 16° C. to 20° C.
   Time to produce a rooted young plant, summer.—About three months at temperatures ranging from about 16° C. to 20° C.
   Root description.—Medium in thickness; fibrous and fleshy; close to 173D in color.
   Rooting habit.—Moderately freely branching; medium density.
Plant description:
   Plant form and growth habit.—Herbaceous perennial; compact, upright and uniformly mounded plant habit; freely clumping habit with leaves arising from underground rhizomes; moderately vigorous growth habit.
   Plant height.—About 40 cm.
   Plant width (spread).—About 50 cm.
Leaf description:
   Length.—About 8 cm.
   Width.—About 8 cm.
   Shape.—Cordate.
   Apex.—Apiculate.
   Base.—Cordate.
   Margin.—Entire; some rippling.
   Texture, upper and lower surfaces.—Pubescent, rough.
   Venation pattern.—Lacinate.
   Color.—Developing leaves, upper surface: Venal areas and margins, close to 129A; intervenal areas, close to 192A. Developing leaves, lower surface: Venal areas and margins, close to 129B; intervenal areas, close to 192D. Fully developed leaves, upper surface: Venal areas and margins, close to 130B; intervenal areas, close to 192C; venation, close to 192A. Fully developed leaves, lower surface: Venal areas and margins, close to 130C; intervenal areas, close to 192D; venation, close to 129D.
   Petioles.—Length: About 25 cm. Diameter: About 2 mm. Texture, upper and lower surfaces: Rough. Color, upper surface: Close to 130B. Color, lower surface: Close to 138C.
Flower description:
   Flower type and flowering habit.—Single campanulate flowers arranged on terminal cymes; usually about 75 to 110 flowers developing per inflorescence during the flowering season; flowers face mostly upright.
   Fragrance.—None detected.
   Natural flowering season.—Plants flower continuously from April until June in the United Kingdom.
   Postproduction longevity.—Flowers last about five days on the plant; flowers not persistent.
   Flower buds.—Height: About 1 mm. Diameter: About 1 mm. Shape: Orbicular. Color: Close to 75D.
   Inflorescence height.—About 40 cm to 45 cm.
   Inflorescence diameter.—About 10 cm.
   Flower diameter.—About 3 mm.
   Flower depth.—About 3 mm.
   Petals.—Quantity per flower: Typically five fused in a single whorl. Length: About 1.5 mm. Width: About 1.5 mm. Shape: Orbicular. Apex: Rounded. Margin: Sagittate. Texture, upper and lower surfaces: Smooth, glabrous. Color: When opening, upper surface: Close to 108A. When opening, lower surface: Close to 108C. Fully opened, upper surface: Close to 108B; color becoming closer to 108D with development. Fully opened, lower surface: Close to 108D; color does not change with development.
   Sepals.—Quantity per flower: Typically five in a single whorl. Length: About 1 mm. Width: About 1 mm. Shape: Orbicular. Apex: Rounded. Base: Cuneate. Margin: Entire. Texture, upper and lower surfaces: Smooth, glabrous. Color, upper surface: Close to 130A. Color, lower surface: Close to 130C.
   Peduncles.—Length: About 2.5 cm to 3 cm. Diameter: About 2 mm. Strength: Moderately strong. Aspect: Upright to about 45° from vertical. Texture: Pubescent. Color: Close to 136C.
   Pedicels.—Length: About 1 mm. Diameter: About 1 mm. Strength: Moderately strong. Aspect: Mostly upright. Texture: Pubescent. Color: Close to 136A.
   Reproductive organs.—Stamens: Quantity per flower: Typically five. Filament length: About 1 mm. Filament color: Close to 130C. Anther shape: Oblong, flattened. Anther length: Less than 1 mm. Anther color: Close to 186D. Pollen amount: Scarce. Pollen color: Close to 173D. Pistils: Quantity per flower: One. Pistil length: About 1 mm. Stigma shape: Tubular. Stigma color: Close to 155D. Style length: About 1 mm. Style color: Close to 155D. Ovary color: Close to 155D.
   Seeds.—Quantity: Few to none. Length: Less than 1 mm. Diameter: Less than 1 mm. Color: Dark brown to almost black.
Disease & pest resistance: Plants of the new Brunnera have not been noted to be resistant to pathogens and pests common to Brunnera.
Garden performance: Plants of the new Brunnera have been observed to have good garden performance and tolerate rain, wind and temperatures ranging from about −20° C. to about 25° C.

It is claimed:

1. A new and distinct Brunnera plant named ‘Litjac’ as illustrated and described.
